SEQUENCE LISTING (5 sequences)

SEQ ID NO: 1 - DNA, 20 bp, Escherichia Coli
cccgagactcagtgaaattg

SEQ ID NO: 2 - DNA, 22 bp, Escherichia coli
cccaagaattcatatcgacggc

SEQ ID NO: 3 - DNA, 27 bp, Escherichia coli
cccaagcttcgctactgccgccaggca

SEQ ID NO: 4 - DNA, 29 bp, Escherichia coli
tcccccgggagtagggaactgccaggcat

SEQ ID NO: 5 - DNA, 20 bp, Escherichia coli
ggctctctttcagacttggg

(19) United States
(12) Patent Application Publication
(10) Pub. No.: US 2010/0087336 A1
(43) Pub. Date: Apr. 8, 2010

(54) FUNCTIONAL NUCLEIC ACIDS AND METHODS

(75) Inventors: George Jackson, Pearland, TX (US); Roger McNichols, Pearland, TX (US); Ulrich Strych, Houston, TX (US); George E. Fox, Manvel, TX (US); Victor G. Stepanov, Houston, TX (US); Yamei Liu, Irving, TX (US)

Correspondence Address: BIOTEX, INC., 8058 EL RIO STREET, HOUSTON, TX, 77054, US

(73) Assignees: BIOTEX, INC., Houston, TX (US); UNIVERSITY OF HOUSTON, Houston, TX (US)

(21) Appl. No.: 12/558,523

(22) Filed: Sep. 12, 2009

Related U.S. Application Data
(63) Continuation-in-part of application No. 12/044,737, filed on Mar. 7, 2008.
(60) Provisional application No. 60/905,792, filed on Mar. 8, 2007.

(51) Int. Cl. C40B 50/06; C12N 15/63; C12N 5/00; C12N 1/00

(52) U.S. Cl. 506/26; 435/320.1; 435/325; 435/243

(57) ABSTRACT

The present invention relates to methods of generating amounts of selective nucleic acids. The present invention further relates to selective nucleic acids incorporated within non-coding nucleic acids, capable of binding to or altering a target molecule. Selective nucleic acids may generally refer to, but are not limited to, deoxyribonucleic acids (DNAs), ribonucleic acids (RNAs), artificially modified nucleic acids, combinations or modifications thereof. Selective nucleic acids may also generally refer to, but are not limited to, nucleic acid aptamers, aptazymes, ribozymes, deoxyribozymes, nucleic acid probes, small interfering RNAs (siRNAs), micro RNAs (miRNAs), short hairpin RNAs (shRNAs), antisense nucleic acids, diagnostic probes or probe libraries, aptamer inhibitors, precursors of any of the above and/or combinations or modifications thereof. In one aspect, a method for generating amounts of selective nucleic acids includes incorporating a selective nucleic acid sequence into a carrier nucleic acid. In general, the carrier nucleic acid may be transcribed by a cell into a product nucleic acid which may carry an incorporated selective nucleic acid sequence.

### CROSS-REFERENCE TO RELATED APPLICATIONS

This application is a continuation-in-part of U.S. utility patent application Ser. No. 12/044,737, filed Mar. 7, 2008, entitled “Functional Nucleic Acids for Biological Sequestration”, which is still pending, which claims the benefit of U.S. provisional patent application Ser. No. 60/905,792, filed Mar. 8, 2007, entitled “Aptamers, ribozymes, and other functional RNAs within non-coding RNAs for biological remediation or concentration”, the contents of all of which are hereby incorporated by reference.

### FIELD OF THE INVENTION

The present invention relates to methods of generating amounts of selective nucleic acids. The present invention further relates to selective nucleic acids incorporated within non-coding nucleic acids, capable of binding to or altering a target molecule.

### BACKGROUND OF THE INVENTION

In recent years RNA has been found to play an increasing number of previously unexpected catalytic and regulatory roles. One of the most interesting of these is the discovery of post-transcriptional gene silencing by short interfering RNA or siRNA, in which 21-23 nucleotide oligoribonucleotides mediate specific cleavage or translational repression of mRNA. Use of exogenous siRNAs to control gene expression or completely “knock out” expression is termed “RNA interference” or RNAi. When originally discovered, RNA interference was thought to be an oddity of the biology of Caenorhabditis elegans, however it has since been generalized to plants and mammalian systems, and may even exist in prokaryotes. Since the discovery and elucidation of the mechanisms of action of siRNA, understanding and applications have advanced such that it is now reasonable to consider large-scale manufacture of specific molecules for the purposes of studying RNAi in model organisms, and for developing therapeutics to important diseases. Unfortunately, the yield and costs associated with in vitro transcription or chemical synthesis of RNA at very large scale are prohibitive.

The field of bioremediation has focused primarily on chemical transformation of contaminants as aided or catalyzed by microbes and/or plants (phytoremediation). However, the effects of complexation, adsorption, absorption, or any process otherwise resulting in sequestration or reduced mobility of contaminants may be just as important for many applications. Organisms have been widely selected and/or modified to aid in the treatment of waste products. The selection and modifications have largely been focused on introducing and/or improving the catalytic capabilities of enzymes for breaking down and/or otherwise transforming wastes and contaminants. Enzymatic mechanisms thus remain the dominant means for treating substances using organisms.

### SUMMARY OF THE INVENTION

The present invention relates to methods of generating amounts of selective nucleic acids. The present invention further relates to selective nucleic acids incorporated within non-coding nucleic acids, capable of binding to or altering a target molecule. Selective nucleic acids may generally refer to, but are not limited to, deoxyribonucleic acids (DNAs), ribonucleic acids (RNAs), artificially modified nucleic acids, combinations or modifications thereof. Selective nucleic acids may also generally refer to, but are not limited to, nucleic acid aptamers, aptazymes, ribozymes, deoxyribozymes, nucleic acid probes, small interfering RNAs (siRNAs), micro RNAs (miRNAs), short hairpin RNAs (shRNAs), antisense nucleic acids, diagnostic probes or probe libraries, aptamer inhibitors, precursors of any of the above and/or combinations or modifications thereof.

In one aspect, a method for generating amounts of selective nucleic acids includes incorporating a selective nucleic acid sequence into a carrier nucleic acid. In general, the carrier nucleic acid may be transcribed by a cell into a product nucleic acid which may carry an incorporated selective nucleic acid sequence. In general, it may be desirable for a cell to transcribe a carrier nucleic acid in relatively large amounts, and it may be further desirable that a carrier nucleic acid may be substantially stable against degradation by the cell and/or other sources of degradation. Further in general, a carrier nucleic acid may include a non-coding nucleic acid (i.e. a nucleic acid that does not encode a protein gene product). The carrier nucleic acid may also be naturally present within a cell or it may be artificially incorporated and/or modified.

In another aspect of the invention, the selective nucleic acids may target any appropriate target molecule, substance, composition, biological target and/or any other appropriate target or combination thereof. In some embodiments, the selective nucleic acids may target, for example, intracellular targets, such as, for more example, other nucleic acids, proteins, cellular structures, signaling molecules and/or any other appropriate intracellular target or combination thereof. In one exemplary embodiment, selective nucleic acids may target messenger RNAs (mRNAs), such as, for example, through an RNA interference (RNAi) mechanism. The present invention further relates to methods for generating and/or improving the interaction of nucleic acids with substances for removal and/or treatment. Bulk volumes may generally refer to any volume of substance wherein the removal and/or treatment of substances therein may occur. In some exemplary embodiments, bulk volumes may include, for example, waste fluid volumes and/or streams, contaminated fluids, and/or any other appropriate form of treatable waste. A bulk volume may also refer, in general, to volumes for filtration, purification, sequestration of particular substances, and/or any other appropriate volume requiring a form of separation.

In one aspect of the present invention, a method for improving the removal and/or treatment of substances in bulk volumes includes generating nucleic acids that may interact with at least one target substance. In general, such nucleic acids may be aptamers.

In some embodiments of this invention, the target molecules are contaminants present in bulk volumes. Bulk volumes include but are not limited to bodily wastes, municipal wastes, industrial effluents, bodies of fresh water, etc. In another embodiment, target molecules are valuable products that need to be reclaimed from bulk volumes. These valuable products include but are not limited to valuable metals, antibodies, drugs, hormones, proteins and pharmaceuticals.

In some embodiments, an expression vector may include a chimeric gene encoding a selective nucleic acid within a non-coding nucleic acid, the selective nucleic acid of which may be capable of binding to or altering target molecules, operatively linked to a functional promoter, where the vector when transfected in a host, such as a cell, transcribes the chimeric gene. Also, disclosed are embodiments of an isolated cell comprising the expression vector described supra. Additionally, disclosed are embodiments of an isolated cell comprising at least one nucleic acid ligand sequence, incorporated into a genomic non-coding nucleic acid, where the nucleic acid ligand sequence binds to or catalytically alters a target molecule.

Provided herein are methods for sequestering within a cell a plurality of target molecules, present in a bulk volume comprising, generating a library of nucleic acid ligand sequences capable of binding to said target molecules; incorporating the nucleic acid ligand sequences in at least one non-coding nucleic acid within a cell; culturing the cell to achieve a cell population; contacting the cell population with the bulk volume; and separating the cell population from the bulk volume.

Furthermore, provided are methods for bioremediation of contaminants present in a bulk volume comprising, generating a library of nucleic acid ligand sequences capable of binding to or altering the contaminants; incorporating the nucleic acid ligand sequences in at least one non-coding nucleic acid in a cell; culturing the cell to achieve a cell population; contacting the cell population with the bulk volume; and separating the cell population from the bulk volume.

In a further aspect, the present invention includes methods of purifying and/or isolating the selective nucleic acids from a host cell, a carrier molecule, and/or both. In some embodiments, enzymatic, catalytic nucleic acid, chemical excision, and/or any other appropriate excision methods or combinations thereof may be utilized to excise selective nucleic acids from a carrier nucleic acid. The selective nucleic acids, the carrier nucleic acids containing the selective nucleic acids, and/or both may also be at least partially selectively purified from a host cell by, for example, partial lysis and/or perforation of a host cell.

In general, aptamers and/or other nucleic acids may be generated to bind with relatively high affinity to a particular substance. Numerous methods of generating aptamers are known in the art. A common method of generating aptamers is known as the Systematic Evolution of Ligands by Exponential Enrichment or SELEX. In general, the process may include the synthesis of a large oligonucleotide library consisting of randomly generated sequences of fixed length flanked by constant 5′ and 3′ ends that may serve as primers. For a randomly generated region of length n, the number of possible sequences in the library is 4n. The sequences in the library may then be exposed to the target substance and those that do not bind the target may be removed, such as by chromatography methods. The bound sequences may then be eluted and amplified by polymerase chain reaction (PCR) to prepare for subsequent rounds of selection in which the stringency of the elution conditions may be increased to identify the strongest-binding sequences. An oligonucleotide library may also omit the constant primer regions, which may be difficult to remove after the selection process due to interactions with the random region, such as, for example, secondary structure stabilization.

The aptamer generation process may be performed in vitro or the process may be performed in vivo. In one aspect, an in vivo aptamer generation may be performed utilizing a host organism. In general, a host organism may be useful in performing the amplification of nucleic acids as such processes are typically innate to all cells. In some exemplary embodiments, prokaryotic hosts such as bacteria may be utilized, as such hosts may typically be easily cultured and/or provide high production of nucleic acids. In other embodiments, eukaryotic hosts may also be utilized.

Nucleic acid sequences may be included in an organism by a variety of methods, such as, for example, transformation of a cell utilizing a nucleic acid construct, such as a plasmid. Nucleic acid sequences may also be incorporated into the nucleic acid sequence of a host organism. The included nucleic acid sequence may contain, aside from containing a nucleic acid sequence with particular binding and/or catalytic activity, other features, such as, for example, selection factors including antibiotic resistance genes, detection assay elements, controllable expression elements, and/or any other appropriate features.

In another aspect, aptamers as discussed above may be utilized as affinity handles for purification. For example, a non-coding nucleic acid may contain a high-affinity aptamer handle as well as a sequence of therapeutic or diagnostic value. The desired high-value nucleic acid may then be readily purified by binding the aptamer portion. Aptamers to common chromatographic matrices such as agarose, Sephadex, Sepharose, as well as more specialized affinity resins with immobilized metals, antibodies, proteins, peptides, and/or any other appropriate affinity material may be utilized. Aptamers to such affinity ligands may be developed by well established in vitro methods or by in vivo methods similar to those discussed above. Inserted aptamers may then be fused to nucleic acids which may be used for therapeutic and/or diagnostic functions, such as, for example, short-interfering RNAs (siRNAs), microRNAs (miRNAs), short-hairpin RNAs (shRNAs), antisense molecules, diagnostic probes or probe libraries, aptamer inhibitors, and/or precursors thereof. Aptamer inhibitors may be developed to many important biological pathways such as G-coupled protein receptors, protein kinases, and/or any other appropriate pathways. The therapeutic nucleic acid with an aptamer affinity handle may also be included in another nucleic acid sequence, such as the degradation resistant sequences and/or high production sequences discussed above. Aptamers may then be utilized as affinity handles for molecules which may then be sequenced, probed by hybridization, and/or characterized by some other analytical technique, such as, for example, mass spectrometry for organism identification.

In still other embodiments, inserted nucleic acid sequences may also be useful for highly specific intracellular labeling and/or cellular signal tracking. For example, an aptamer may include a fluorescent- and/or radio-label could be concatenated and/or fused to an aptamer targeting a particular cellular component, such as an important protein, enzyme, organelle, and/or any other appropriate component. This aptamer fusion may then be expressed at high levels within a non-coding nucleic acid, as described above. Cells expressing such aptamers may thus have a built-in ability to monitor specific cellular processes.

The present invention together with the above and other advantages may best be understood from the following detailed description of the embodiments of the invention illustrated in the drawings.

### BRIEF DESCRIPTION OF THE FIGURES

FIG. 1 illustrates an example generating amounts of selective nucleic acids;

FIG. 2 illustrates excision of an insert nucleic acid from a carrier nucleic acid;

FIG. 3 shows a gel of purification products of DNAzyme digestion of an aRNA;

FIG. 3a shows a table of yields of purification with DNAzyme digestion of an aRNA;

FIG. 4 shows a gel of RNase III digestion of an aRNA; and

FIG. 5 shows a gel of RNase H digestion of an aRNA.

### DETAILED DESCRIPTION OF THE INVENTION

The detailed description set forth below is intended as a description of the presently exemplified device provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to methods of generating amounts of selective nucleic acids. The present invention further relates to selective nucleic acids incorporated within non-coding nucleic acids, capable of binding to or altering a target molecule. Selective nucleic acids may generally refer to, but are not limited to, deoxyribonucleic acids (DNAs), ribonucleic acids (RNAs), artificially modified nucleic acids, combinations or modifications thereof. Selective nucleic acids may also generally refer to, but are not limited to, nucleic acid aptamers, aptazymes, ribozymes, deoxyribozymes, nucleic acid probes, small interfering RNAs (siRNAs), micro RNAs (miRNAs), short hairpin RNAs (shRNAs), antisense nucleic acids, diagnostic probes or probe libraries, aptamer inhibitors, precursors of any of the above, and/or combinations or modifications thereof. Selective nucleic acids may also include, for example, selective nucleic acid ligands. In general, when referring to a selective nucleic acid, it may be understood that the selective nucleic acid may refer to the sequence of a selective nucleic acid, its complementary sequence, a product nucleic acid of such a sequence, a gene encoding such a product nucleic acid, and/or a combination or modification thereof.

An “aptamer” refers to a nucleic acid molecule that is capable of binding to a particular molecule of interest with high affinity and specificity (Tuerk and Gold, Science 249:505 (1990); Ellington and Szostak, Nature 346:818 (1990). The binding of a ligand to an aptamer, which is typically RNA, may also change the conformation of the aptamer and the nucleic acid within which the aptamer is located. The conformation change inhibits translation of an mRNA in which the aptamer is located, for example, or otherwise interferes with the normal activity of the nucleic acid. This type of interaction, with a small molecule metabolite, for example, coupled with subsequent changes in nucleic acid function has been referred to as a ‘riboswitch’. Aptamers may also be composed of DNA or may comprise non-natural nucleotides and nucleotide analogs. The method of selection may be by, but is not limited to, affinity chromatography and the method of amplification by reverse transcription (RT) or polymerase chain reaction (PCR).

Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand as compared to the dissociation constant of the aptamer for other materials in the environment or unrelated molecules in general. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less.

An aptamer will typically be between about 10 and about 300 nucleotides in length. More commonly, an aptamer will be between about 30 and about 100 nucleotides in length.

The terms “nucleic acid molecule” and “polynucleotide” refer to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Cassol et al. (1992); Rossolini et al., Mol. Cell. Probes 8:91-98 (1994)). Also included are molecules having naturally occurring phosphodiester linkages as well as those having non-naturally occurring linkages, e.g., for stabilization purposes. The nucleic acid may be in any physical form, e.g., linear, circular, or supercoiled. The term nucleic acid is used interchangeably with oligonucleotide, gene, cDNA, and mRNA encoded by a gene.

A riboswitch is typically considered a part of an mRNA molecule that can directly bind a small target molecule, and whose binding of the target affects the gene's activity [Tucker B J, Breaker R R (2005). “Riboswitches as versatile gene control elements”. Curr Opin Struct Biol 15 (3): 342-8]. Thus, an mRNA that contains a riboswitch is directly involved in regulating its own activity, depending on the presence or absence of its target molecule. By definition, then, a riboswitch has a region of aptamer-like affinity for a separate molecule. Thus, in the broader context of the instant invention, any aptamer included within a non-coding nucleic acid could be used for sequestration of molecules from bulk volumes. Downstream reporting of the event via “(ribo)switch” activity may be especially advantageous. A similar concept is coined by the phrase “aptazyme” in which an aptamer region is used as an allosteric control element and coupled to a region of catalytic RNA (a “ribozyme” as described below).

A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome. More recently it has been shown that catalytic RNAs can be “evolved” by in vitro methods [1. Agresti J J, Kelly B T, Jaschke A, Griffiths A D: Selection of ribozymes that catalyse multiple-turnover Diels-Alder cycloadditions by using in vitro compartmentalization. Proc Natl Acad Sci USA 2005, 102:16170-16175; 2. Sooter L J, Riedel T, Davidson E A, Levy M, Cox J C, Ellington A D: Toward automated nucleic acid enzyme selection. Biological Chemistry 2001, 382(9):1327-1334.]. Winkler et al. have shown [Winkler W C, Nahvi A, Roth A, Collins J A, Breaker R R: Control of gene expression by a natural metabolite-responsive ribozyme. Nature 2004, 428:281-286.] that, similar to riboswitch activity discussed above, ribozymes and their reaction products can regulate gene expression. In the context of the instant invention, it may be particularly advantageous to place a catalytic RNA or ribozyme within a larger non-coding RNA such that the ribozyme is present at many copies within the cell for the purposes of chemical transformation of a molecule from a bulk volume. Furthermore, encoding both aptamers and ribozymes in the same non-coding RNA may be particularly advantageous.

The term “gene” is used broadly to refer to any segment of DNA associated with a biological function. Thus, genes include coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “bases” refers to both the deoxyribonucleic and ribonucleic acids. The following abbreviations are used, “A” refers to adenine as well as to its deoxyribose derivative, “T” refers to thymine “U” refers to uridine, “G” refers to guanine as well as its deoxyribose derivative, “C” refers to cytosine as well as its deoxyribose derivative. A person having ordinary skill in this art would readily recognize that these bases may be modified or derivatized to optimize the methods of the present invention.

In one aspect, a method for generating amounts of selective nucleic acids includes incorporating a selective nucleic acid sequence into a carrier nucleic acid. In general, the carrier nucleic acid may be transcribed by a cell into a product nucleic acid which may carry an incorporated selective nucleic acid sequence. In general, it may be desirable for a cell to transcribe a carrier nucleic acid in relatively large amounts, and it may be further desirable that a carrier nucleic acid may be substantially stable against degradation by the cell and/or other sources of degradation. Further in general, a carrier nucleic acid may include a non-coding nucleic acid (i.e. a nucleic acid that does not encode a protein gene product). The carrier nucleic acid may also be naturally present within a cell or it may be artificially incorporated and/or modified.

Some nucleic acids, such as for example RNAs, may be subject to rapid degradation in biological environments due to targeting by nuclease activity and/or other environmental factors. However, some nucleic acid sequences may be resistant to such degradation either by their inherent size and/or their similarity to structured RNAs within the cell. A degradation resistant nucleic acid sequence may be utilized to protect a selective nucleic acid sequence from degradation in an organism and/or other biological environment. The selective nucleic acid sequence may be inserted, for example, into an appropriate region of the degradation-resistant nucleic acid sequence, the product of which may generally be referred to as an artificial RNA (aRNA). The degradation-resistant nucleic acid may be, for further example, subject to a high degree of molecular production, such as, for example, a ribosomal RNA (rRNA). In addition to rRNA, selective nucleic acids may also be inserted in other non-coding RNAs such as, for example, RNase P, tRNAs, small nuclear RNA (snRNA), small nucleolar RNAs (snoRNAs), efference RNA (eRNA), tmRNA, and/or any other appropriate nucleic acids which may be “non-coding”. These nucleic acids, while non-coding, may include the capacity to accrue to significant levels within cells and thus may be useful for high production of selective nucleic acids contained within the non-coding nucleic acids. In general, a region of a carrier nucleic acid may be utilized that may generally not disrupt the production and/or accrual of the carrier nucleic acid in a host cell. Further in general, a region of a carrier nucleic acid which may be tolerant to an inserted sequence, such as by not creating instability or targeting the carrier for degradation, may be utilized for the insertion of a selective nucleic acid sequence.

Nucleic acid sequences may be introduced in an organism by a variety of methods, such as, for example, transformation of a cell utilizing a nucleic acid construct, such as a plasmid. Nucleic acid sequences may also be incorporated into the host chromosome. The included nucleic acid sequence may contain, for example, a nucleic acid sequence with particular binding and/or catalytic activity, and/or other features, such as, for example, selection factors including antibiotic resistance genes, detection assay elements, controllable expression elements, and/or any other appropriate features.

A host organism may be useful in performing the amplification of nucleic acids as such processes are typically innate to all cells. Prokaryotic hosts such as bacteria or eukaryotic hosts may be utilized. The criteria for selection of a host organism may include the ability to be easily cultured or grown as well as provide high production of nucleic acids. Examples of host organisms may include, but are not limited to, E. coli, Staphylococcus, Bacillus, Pseudomonas, Citrobacteia, Klebsilla, Rhodococcus, and/or any other appropriate organism, such as any number of eubacteria, archaea, fungi, plant, and/or mammalian cells. Combinations of organism hosts may also be utilized.

In another aspect of the invention, the selective nucleic acids may target any appropriate target molecule, substance, composition, biological target and/or any other appropriate target or combination thereof. In some embodiments, the selective nucleic acids may target, for example, intracellular targets, such as, for more example, other nucleic acids, proteins, cellular structures, signaling molecules and/or any other appropriate intracellular target or combination thereof.

In some exemplary embodiments, selective nucleic acids may target messenger RNAs (mRNAs), such as, for example, through an RNA interference (RNAi) mechanism. The selective nucleic acids may be, for example, siRNAs, miRNAs, shRNAs, antisense nucleic acids, precursors thereof and/or any other appropriate nucleic acid for participation in an RNAi mechanism. In general, RNAi mechanisms may function by binding a short RNA molecule which may be complementary to at least a portion of an mRNA in a complex called the RNA Induced Silencing Complex (RISC). The RISC then degrades the targeted mRNA which may generally result in decreased expression of the product of the mRNA, which may be a protein. This method of decreasing expression may generally be referred to as “silencing” a gene. The RNAi mechanism may further include processing of a selective nucleic acid into an active molecule, such as a precursor of an siRNA or miRNA into an active siRNA or miRNA. This may generally be performed by the enzyme DICER, an RNA endonuclease which may cleave a pre-miRNA/siRNA stem-loop or a double stranded RNA (dsRNA) into a 20- to 25-base-pair double-stranded RNA fragment with a 2 nucleotide 3′ overhang at each end.

In some exemplary embodiments, selective nucleic acids that may participate in an RNAi mechanism may be included in a carrier nucleic acid, such as for example a non-coding nucleic acid. In some embodiments, the selective nucleic acid may participate in an RNAi mechanism of, for example, a desired cell, cell type, organism, organism type and/or any other appropriate set or subset of cells. In some embodiments, it may be desirable for the host cell that may generate such selective nucleic acids to be substantially unresponsive and/or untargeted by the selective nucleic acid and/or associated RNAi mechanism. For example, a host cell may be utilized to generate an amount of a selective nucleic acid which may then be utilized to participate in an RNAi mechanism of another cell type. Further for example, it may be desirable to utilize a high-growth and/or -production rate host cell, such as for example, prokaryotic and/or high-growth eukaryotic cells, to generate selective nucleic acids which may be utilized in other cell types, such as, for example, in therapeutic or diagnostic applications, such as, for example on eukaryotic cells. It may further be desirable to utilize a host cell which may be substantially dissimilar to the cell type that the selective nucleic acid may be utilized on, as this may, for example, aid in reducing undesirable interactions between the host cell and the selective nucleic acid.

In other embodiments, it may be desirable for the host cell to be targeted and/or be substantially responsive to the selective nucleic acid and/or the associated RNAi mechanism.

In one aspect, the selective nucleic acid sequence may be incorporated in an expression vector. In some embodiments, the expression vector may include a chimeric gene encoding selective nucleic acids within a carrier nucleic acid, such as a non-coding nucleic acid, which may be further operatively linked to a functional promoter, where the vector when transfected and/or otherwise introduced into a host may transcribe the chimeric gene, and where the gene product may be capable of binding to or altering target molecules. In an embodiment, the vector may further include a selection marker. Specifically, the selection marker may be an antibiotic resistance marker. Moreover, the promoter may be a T7 RNA polymerase or a ribosomal RNA promoter. In some embodiments, the selective nucleic acid may be any of the selective nucleic acids discussed above. Additionally, the carrier nucleic acid, such as non-coding nucleic acid, may be selected from the group consisting of rRNA, tRNA, RNAase P, small nuclear RNA (snRNA), small nucleolar RNA (snoRNA), efference RNA (eRNA) and tmRNA. In embodiments incorporating a selective nucleic acid sequence into an rRNA, the 5S, 16S and/or 23S rRNAs may be utilized. An isolated cell including the expression vector described supra may also be utilized. In general, the cell may be a prokaryotic cell or a eukaryotic cell.

In another embodiment, an isolated cell may include at least one selective nucleic acid sequence, incorporated into a carrier nucleic acid, such as a genomic non-coding nucleic acid, where the nucleic acid ligand sequence may bind to or catalytically alter a target molecule, such as by participation in an RNAi mechanism. In general, the selective nucleic acid sequence may be incorporated into the genomic non-coding nucleic acid by standard molecular biology methods, such as, for example, homologous recombination, and/or any other appropriate method. The non-coding nucleic acid may further be selected from the group consisting of rRNA, tRNA, RNAase P, small nuclear RNA (snRNA), small nucleolar RNA (snoRNA), efference RNA (eRNA) and tmRNA. In general, the cell may be a prokaryotic cell or a eukaryotic cell.

It may generally be desirable to utilize specific and/or modified host cells for generating selective nucleic acids. For example, host cells deficient and/or lacking in at least one type of functional nuclease, such as, for further example, an RNase III-minus strain, may be desirable to minimize any host cell degradation of the desired selective nucleic acid. For further example, a host cell with controllable nucleases, such as, for example, an inserted inducible RNase III gene, may also be utilized such that the nuclease may be deactivated during production of the selective nucleic acid.

Nucleic acid sequences with established and/or otherwise known ability to bind and/or otherwise interact with a target substance may also be utilized as a starting sequence. For example, known, well-characterized aptamers to ions, small inorganic or organic molecules, proteins, peptides, whole viral particles, and/or other appropriate targets may be inserted into non-coding nucleic acid sequences for the purpose of, for example, sequestering such molecular targets. Such aptamers, identified by in vitro methods of selection have different binding affinity within the context of the surrounding non-coding nucleic acid, but they nevertheless retain significant affinity for their targets. Upstream (e.g. 5′-) and downstream (e.g. 3′-)polynucleotide spacers and/or appendages can be added to the known sequences to relax conformational constraints placed upon the sequences by tethering them within the context of the non-coding nucleic acid. Furthermore, multiple aptamers are concatenated to give increased avidity to the molecular target(s).

In a further aspect, the present invention includes methods of purifying and/or isolating the selective nucleic acids from a host cell, a carrier molecule, and/or both. The selective nucleic acids, the carrier nucleic acids containing the selective nucleic acids, and/or both may be at least partially selectively purified from a host cell by, for example, partial lysis and/or perforation of a host cell. Also, in embodiments where the selective nucleic acid sequence may be incorporated into a particular carrier nucleic acid, recovery of the aRNA may be accomplished by, for example, partially lysing and/or selectively eluting a particular size of nucleic acid which may generally correspond to the size of the desired aRNA.

In some embodiments, enzymatic, catalytic nucleic acid, chemical excision, and/or any other appropriate excision methods or combinations thereof may be utilized to excise selective nucleic acids from a carrier nucleic acid.

In some embodiments, ribonucleases, such as, for example, ribonuclease III (RNase III) may be utilized. RNase III may generally cleave dsRNAs and/or RNA “hairpins” of sufficient length and may be utilized to cleave the inserted selective nucleic acid from the carrier nucleic acid. For another example, RNase H may also be utilized. RNase H may generally degrade RNAs hybridized to complementary DNA. Complementary DNAs may thus be hybridized to the carrier nucleic acid which may be an RNA. RNase H may then be utilized to degrade the carrier nucleic acid which may leave the selective nucleic acid for further purification.

In other embodiments, deoxyribozymes (DNAzymes) may be utilized. DNAzymes may generally be DNAs with catalytic activity and may recognize particular sequences of other nucleic acids to mediate sequence-dependent cleavage. Further in general, DNAzymes may be at least partially complementary to a target nucleic acid, such as an RNA, and may be utilized to hybridize to the RNA in a sequence dependent manner. The DNAzymes may then cleave the target nucleic acid, such as, for example, through metal ion (e.g. magnesium ions) dependent cleavage. Any appropriate DNAzyme, such as, for example, 8-17 and/or 10-class DNAzymes may be utilized. Sequence motifs may further be included with the selective nucleic acid sequence, such as, for example, flanking the desired sequence, such that the DNAzymes may hybridize to the sequence motifs and be utilized to excise the selective nucleic acid.

In other embodiments, the selective nucleic acid sequence may be self-cleaving. For example, cis-cleaving ribozymes (RNAzymes) may be included with the selective nucleic acid sequence, such as, for example, flanking the desired sequence, such that the RNAzymes may be utilized to excise the selective nucleic acid. In general, the self-excision may be controllable and/or triggerable such that the selective nucleic acid is not excised prematurely.

In other embodiments, chemical excision methods may also be utilized. “Chemical scissors,” such as DNA molecules containing acridine residues at the cleavage sites may be utilized, such as by hybridization to the carrier nucleic acid and/or the selective nucleic acid. A lanthanoid salt, such as lutetium chloride may further be utilized to induce transesterification to affect cleavage. It may generally be understood that a single excision method, combinations, modifications, and/or any other appropriate methods may be utilized to excise the selective nucleic acid from the carrier nucleic acid.

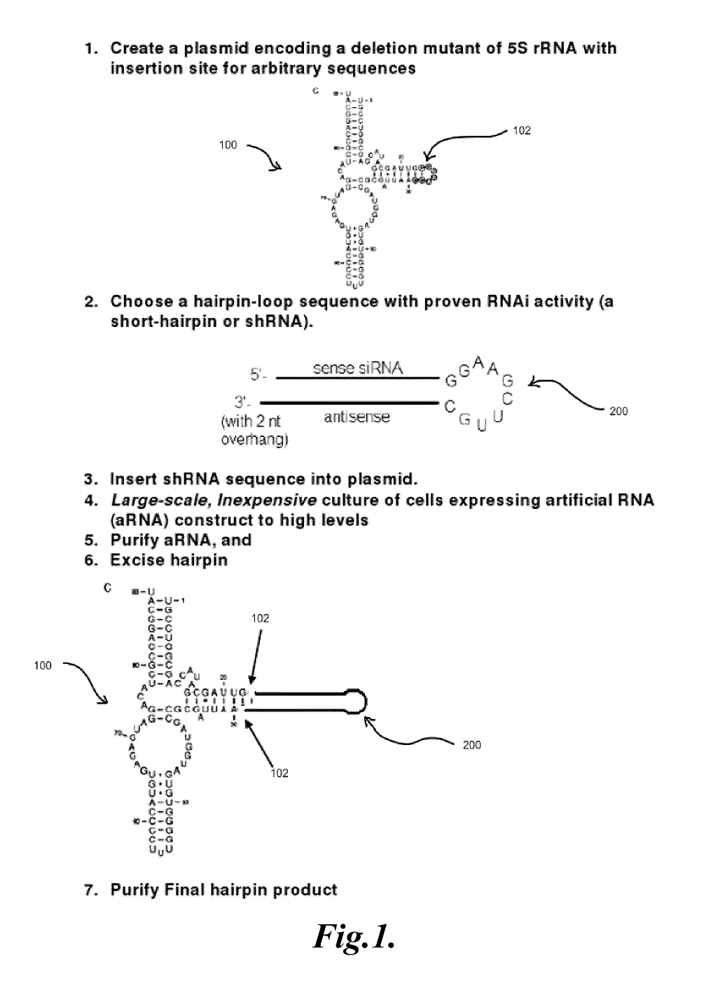

FIG. 1 illustrates an example of a method for generating amounts of a selective nucleic acid. In one embodiment, as illustrated, at a step 1, a plasmid may be generated that may include a deletion mutant 100 of a 5S rRNA with an insertion site 102 which may be utilized to incorporate a nucleic acid sequence such as, for example, a selective nucleic acid sequence. In step 2, a selective nucleic acid sequence, such as an shRNA 200, may be chosen, such as for RNAi activity. At step 3, the sequence to be incorporated, such as the shRNA 200, may be incorporated into the insertion site of the deletion mutant 100, such as by standard molecular biology techniques or methods. The plasmid may then be incorporated into a host cell, such as a relatively inexpensive, scalable cell culture (e.g. E. coli). The culture may then be cultured to produce more host cells which may generally be expressing the aRNA which may include the deletion mutant 100 and the incorporated sequence 200 at step 4. The aRNA may then be purified from the culture, at step 5. The incorporated sequence may then be excised, such as illustrated in step 6 with the deletion mutant 100 and the incorporated shRNA 200 at the insertion sites 102. This may utilize any appropriate excision method, such as those described above. In some embodiments, the incorporated sequence may also contain multiple sequences. This may be desirable, for example, to improve yield by producing more of the desired nucleic acid molecule per carrier nucleic acid. Then at step 7, the final product, such as the shRNA may be purified as a product.

In another aspect, the invention includes a novel methodology to sequester trace contaminants or target molecules from water and other process streams during biological treatment. More specifically, disclosed herein are methods of improving the removal or treatment of target molecules or contaminants in bulk volume by nucleic acid ligands generated to specifically bind to or alter target molecules or contaminants in bulk volumes. Genomic manipulation and selection of prokaryotic or eukaryotic cells will be used to place these nucleic acid ligands into naturally amplified non-coding nucleic acid sequences. By growing cells expressing random nucleic acid sequences, under increasing contaminant concentration, nucleic acid ligands will be selected in vivo that sequester these contaminants.

Bulk volumes refers to any volume of substance wherein the removal and/or treatment of substances therein occurs. Bulk volume includes waste fluids and/or streams, municipal waste and/or any other form of treatable waste.

Target molecules contemplated include but are not limited to metal ions, organic molecules, viral particles, biological molecules, such as antibodies, proteins, enzymes, pharmaceuticals and/or any other substance to be removed and/or treated from a bulk volume. In particular, wastes and contaminants are contemplated. Sequestration of target molecules refers to binding to or altering the target molecules.

Nucleic acid sequences, that can be utilized as discussed include but are not limited to aptamers, ribozymes, aptazymes, riboswitches, and/or any other nucleic acid sequence with particular binding and/or catalytic activity. For example, catalytic nucleic acids may be utilized to perform a treatment reaction, such as degradation, of a target molecule. Catalytic nucleic acids may also augment the catalytic action of other catalytic mechanisms, such as enzymatic catalytic mechanisms in cells.

Aptamers and/or other nucleic acids are generated to bind with relatively high affinity to a target molecule. Numerous methods of generating aptamers are known in the art. A common method of generating aptamers is known as the Systematic Evolution of Ligands by Exponential Enrichment or SELEX. In general, the process includes the synthesis of a large oligonucleotide library consisting of randomly generated sequences of fixed length flanked by constant 5′ and 3′ ends that serve as primers. For a randomly generated region of length n, the number of possible sequences in the library is 4ⁿ. The sequences in the library is then exposed to the target molecule and those that do not bind to the target are removed, such as by chromatography methods. The bound sequences are then eluted and amplified by polymerase chain reaction (PCR) to prepare for subsequent rounds of selection in which the stringency of the elution conditions are increased to identify the strongest-binding sequences.

The aptamer generation process can be performed in vitro or, in some exemplary embodiments, the process may be performed in vivo. An in vivo aptamer generation is performed utilizing a host organism. A host organism is useful in performing the amplification of nucleic acids as such processes are typically innate to all cells. Prokaryotic hosts such as bacteria or eukaryotic hosts are utilized. The criteria for selection of a host organism include ability to be easily cultured or grown as well as provide high production of nucleic acids. Examples of host organisms may include, but are not limited to, E. coli, Staphylococcus, Bacillus, Pseudomonas, Citrobacteia, Klebsilla, Rhodococcus, and/or any other appropriate organism, such as any number of eubacteria, archaea, fungi, plant, and/or mammalian cells. Combinations of organism hosts can also be utilized. The selection of an organism and/or combination of organisms is based on known and/or desirable interaction with a given application and/or target substance. For example, a number of organisms have been typically employed for bioremediation having natural catalytic properties for breakdown of specific contaminant types.

In some exemplary embodiments, a host organism is utilized to both evolve and/or produce a nucleic acid sequence with particular binding and/or catalytic activity. Cells containing a particular nucleic acid sequence are exposed to given concentrations of a target substance. Cells are then selected for a given reaction to the target substance, such as, for example, survival after exposure, and are further selected utilizing increasing concentrations of the substance. This method of selecting cells capable of generating and evolving a nucleic acid sequence is similar to in vitro SELEX. This method can be used for large groupings of different sequences for high-throughput.

Bulk volumes can be treated with the genetically modified cells containing functional aptamer and/or catalytic nucleic acids within non-coding nucleic acid sequences. The genetically modified cells treat, remove and/or sequester target molecules in the bulk volume. The presence of a high concentration of binding and/or catalytic nucleic acids inside the cell creates an equilibrium shift in the bulk volume whereby a given substance is removed from the bulk volume and sequestered in the cell by binding to and/or catalytic action by the modified nucleic acids. The sequestration and/or catalytic action generally constantly removes the targeted molecule from the equilibrium, resulting in a constant influx of the target molecule into the cell. The genetically modified cells, harboring the sequestered target molecules, are then removed from the bulk volume. Appropriate methods of removal of the genetically modified cells include, but are not limited to, filtration, sedimentation, centrifugation (accelerated sedimentation), flocculation, adsorption, membrane filtration, biofilm formation, membrane bioreactor, and/or any other physical configuration otherwise known in the art as a bioreactor, used to separate the treated waste stream from the cells.

Such bioreactors also include in situ remediation techniques in which the genetically modified cells are released into a controlled volume of the environment. Sequestration and/or chemical transformation of contaminants then occurs before the controlled volume passes into another portion of the environment. This is particularly useful in examples where the cells are introduced into waste water and/or other waste streams which are in contact with the environment. The genetically modified cells can be immobilized for contact with a bulk volume while not being distributed into the volume. Immobilization techniques include but are not limited to, microbial mats, mineral amendments, polymer gel formulations, and/or any other appropriate immobilization technique or combination may be utilized. Genetically-modified cells can be tagged for identification such that they can be isolated from a particular environment. Additionally, the cells can be genetically modified to include features for their removal from an environment, such as, for example, a susceptibility factor to a particular substance, an affinity to a particular separation method, and/or any other appropriate removal method.

Further, the cells may also include features for increasing the sequestration rate of a substance in a bulk volume. For example, a molecular channel and/or transporter may be utilized to enhance transport of a substance across the cell membrane into the cell. Metal ion and/or other small ion transport molecules are known and can be incorporated by genetic modification of the cell. Additionally, the cells can be engineered to export the aptamer containing nucleic acids into bulk environment, for example, by including nucleic acid sequences encoding viral packaging and/or export signals. Reuptake of released nucleic acid bound to the target molecule can be engineered for example, by binding to cell surface receptors and/or any other appropriate method.

An aptamer expressed within the context of a larger non-coding nucleic acid can also be used to sequester a valuable substance. For example, copper obtained by “microbial leaching” accounts for more than 15 percent of the annual U.S. copper production. Genetically modified cells bearing aptamers capable of binding target molecules of value, as discussed above, can be utilized to sequester large amounts of valuable metals, hormones, biological drugs, and/or any other appropriate substance. The cells can be concentrated into a biosolid containing a large amount and/or concentration of a given substance.

Aptamers as discussed above may be utilized as affinity handles for purification. For example, a non-coding nucleic acid may contain a high-affinity aptamer handle as well as a sequence of therapeutic or diagnostic value. The desired high-value nucleic acid may be readily purified by binding the aptamer portion. Aptamers to common chromatographic matrices such as agarose, Sephadex, Sepharose, as well as more specialized affinity resins with immobilized metals, antibodies, proteins, peptides, and/or any other appropriate affinity material may be utilized. Aptamers to such affinity ligands may be developed by well established in vitro methods or by in vivo methods similar to those discussed above. Inserted aptamers fused to nucleic acids may be used for therapeutic and/or diagnostic functions, such as, for example, short-interfering RNAs (siRNAs), microRNAs (miRNAs), short-hairpin RNAs (shRNAs), antisense molecules, diagnostic probes or probe libraries, aptamer inhibitors, precursors of all of the above, and/or combinations or modifications thereof. Aptamer inhibitors may be developed to many important biological pathways such as G-coupled protein receptors, protein kinases, and/or any other appropriate pathways. The therapeutic nucleic acid with an aptamer affinity handle may be included in another nucleic acid sequence, such as the degradation resistant sequences and/or high production sequences discussed above. Aptamers utilized as affinity handles for molecules may be sequenced, probed by hybridization, and/or characterized by some other analytical technique, such as, for example, sequencing or mass spectrometry for organism identification.

Inserted nucleic acid sequences are also useful for highly specific intracellular labeling and/or cellular signal tracking. For example, an aptamer including a fluorescent- and/or radio-label can be concatenated and/or fused to an aptamer targeting a particular cellular component, such as an important protein, enzyme, organelle, and/or any other appropriate component. This aptamer fusion can be expressed at high levels within a non-coding nucleic acid, as described above. Cells expressing such aptamers may thus have a built-in ability to monitor specific cellular processes.

In another embodiment, an initial randomized library may be inserted in a non-coding nucleic acid sequence and selected in vitro for certain interaction and/or catalytic activity while contained within a reverse phase emulsion, a method referred to as in vitro compartmentalization. In such a process, on average one template encoding an aptamer would be accommodated within the reverse phase micelle. For those micelles in which the desired catalytic activity was achieved, an affinity handle, such as biotin, may be attached to the encoding gene. Catalytic ribozymes, aptazymes, and/or other catalytic nucleic acids may then be encoded within the context of a larger gene encoding a non-coding nucleic acid, such as rRNA. Affinity handles may then be added to those genes, which may be individually compartmentalized in micelles, which encode nucleic acids with the desired catalytic activity.

Hence, disclosed herein is an expression vector comprising a chimeric gene encoding selective nucleic acid ligands within a non-coding nucleic acid, operatively linked to a functional promoter, where the vector when transfected into a host transcribes the chimeric gene, and where the gene product is capable of binding to or altering target molecules. In an embodiment, the vector further comprises a selection marker. Specifically, the selection marker is an antibiotic resistance marker. Moreover, the promoter is a T7 RNA polymerase or a ribosomal RNA promoter. In general, the nucleic acid ligands are an aptamer, ribozyme, aptazyme or a riboswitch. Additionally, the non-coding nucleic acid is selected from the group consisting of rRNA, tRNA, RNAase P, small nuclear RNA (snRNA), small nucleolar RNA (snoRNA), efference RNA (eRNA) and tmRNA. In general, the target molecules are waste water contaminants. Specifically, the waste water contaminants are inorganic molecules, organic molecules, toxins, proteins, peptides, or viral particles. In one embodiment, the target molecules are hormones, antibodies, proteins, enzymes, pharmaceuticals or valuable metals. Further disclosed is an isolated cell comprising the expression vector described supra. In general, the cell is a prokaryotic cell or a eukaryotic cell.

In another embodiment of the present invention there is an isolated cell comprising at least one nucleic acid ligand sequence, incorporated into a genomic non-coding nucleic acid, where the nucleic acid ligand sequence binds to or catalytically alters the target molecule. In general, the nucleic acid ligand sequence is incorporated into the genomic non-coding nucleic acid by homologous recombination. The non-coding nucleic acid is selected from the group consisting of rRNA, tRNA, RNAase P, small nuclear RNA (snRNA), small nucleolar RNA (snoRNA), efference RNA (eRNA) and tmRNA. In general, the cell is a prokaryotic cell or a eukaryotic cell. Moreover, the nucleic acid sequence is an aptamer, ribozyme, aptazyme or a riboswitch. In general, the target molecule is a waste water contaminant or substance otherwise desired to be sequestered from the treatment stream. Specifically, the waste water contaminants are inorganic molecules, organic molecules, toxins, proteins, peptides, or viral particles. In a related embodiment the target molecule is a hormone, antibody, protein, enzyme, or a valuable metal.

In yet another embodiment, there is provided a method for sequestering within a cell a plurality of target molecules, present in a bulk volume comprising, generating a library of nucleic acid ligand sequences capable of binding to the target molecules; incorporating the nucleic acid ligand sequences into at least one non-coding nucleic acid within a cell; culturing the cell to achieve a cell population; Contacting the cell population with the bulk volume; and separating the cell population from the bulk volume. The method further comprises recovering the target molecule from the cell population. In general, the target molecules are inorganic molecules, organic molecules, toxins, proteins, peptides, and viral particles. In a related embodiment, the target molecules are hormones, antibodies, proteins, enzymes, pharmaceuticals or valuable metals. In general, the separation is accomplished by a method selected from the group consisting of filtration, sedimentation, flocculation, adsorption, membrane filtration, biofilm formation and membrane bioreactor interaction. The non-coding nucleic acid is selected from the group consisting of rRNA, tRNA, RNAase P, small nuclear RNA (snRNA), small nucleolar RNA (snoRNA), efference RNA (eRNA) and tmRNA. Specifically, the incorporation of nucleic acid ligand sequence is into the genomic non-coding nucleic acid. The nucleic acid sequence is an aptamer, ribozyme, aptazyme or a riboswitch. Moreover, the cell is a prokaryotic or a eukaryotic cell.

In yet another embodiment of the present invention, there is provided a method for bioremediation of contaminants present in a bulk volume comprising, generating a library of nucleic acid ligand sequences capable of binding to or altering the contaminants; incorporating the nucleic acid ligand sequences in at least one non-coding nucleic acid in a cell; culturing the cell to achieve a cell population; contacting the cell population with the bulk volume; and separating the cell population from the bulk volume. In general, the contaminants are inorganic molecules, organic molecules, toxins, proteins, peptides, and viral particles. The bulk volume is bodily waste fluids, municipal waste water or effluent from an industrial process.

Accordingly, various embodiments of the present invention disclose an expression vector comprising: a chimeric gene encoding selective nucleic acid ligands within a non-coding nucleic acid, operatively linked to a functional promoter, wherein said expression vector when transfected into a host transcribes said chimeric gene, and wherein said gene product is capable of binding to or altering at least one target molecule.

Further embodiments disclose an isolated cell comprising: at least one nucleic acid ligand sequence incorporated into a genomic DNA encoding a non-coding nucleic acid, wherein said nucleic acid ligand sequence binds to or catalytically alters a target molecule.

Yet further embodiments disclose a method for sequestering within a cell a plurality of target molecules, present in a bulk volume comprising: generating a library of nucleic acid ligand sequences capable of binding to said target molecules; incorporating said nucleic acid ligand sequences into at least one non-coding nucleic acid within a cell; culturing said cell to achieve a cell population; contacting said cell population with said bulk volume; and separating said cell population from said bulk volume.

Various other embodiments disclose methods for bioremediation of at least one contaminant present in a bulk volume comprising generating a library of nucleic acid ligand sequences capable of binding to or altering said at least one contaminant; incorporating said nucleic acid ligand sequences in at least one non-coding nucleic acid in a cell; culturing said cell to achieve a cell population; contacting said cell population with said bulk volume; and, separating said cell population from said bulk volume.

The following examples are given for the purpose of illustrating various embodiments of the invention and are not meant to limit the present invention in any fashion.

### Example 1

### Chromosomal Modification for Encoding Randomized Sequences within Modified rRNA

A plasmid-based system for expressing random libraries of RNA sequences within the context of a larger 5S rRNA sequence will be used. While this system has some advantages in terms of being selectively inducible by IPTG and can be used to readily identify new aptamers, the desired strains should be chromosomal variants. This is mainly because for the water or solids waste applications, it would be undesirable to maintain a plasmid system by the continual addition of an antibiotic. Described below are steps to create a very similar system residing on the chromosome of E. coli.

In order to introduce the necessary genetic modifications the protocol as described in Ammons et al. will be followed. See Ammons D, Rampersad J, Fox G E: A Genomically Modified Marker Strain of Escherichia coli. Current Microbiology 1998, 37:341-346. The aRNAs containing the random RNA libraries within the 5S rRNA will be subcloned into the plasmid containing the recombination cassette, pKO3-SARP. The host strain for integration of the artificial RNA will be the recombination proficient E. coli strain EMG2 (F′, lambda+). Excision of the kanamycin cassette will be performed with the aid of the Saccharomyces cerevisiae gene coding for Flp recombinase (FLP), contained in the pCP20 plasmid, and the Flp DNA target sequence (FRT), present in pK03-SARP.

### Example 2

### Chromosomal Integration of the Randomized aRNA Library

The protocol to be used for gene replacement will be as described in Ammons et al. See Ammons D, Rampersad J, Fox GE: A Genomically Modified Marker Strain of Escherichia coli. Current Microbiology 1998, 37:341-346, which has been derived from Link et al. See Link et al Methods for Generating Precise Deletions and Insertions in the Genome of Wild-Type Escherichia coli: Application to Open Reading Frame Characterization. Journal of Bacteriology 1997, 179(20):6228-6237. Briefly, EMG2 cells are transformed with the pKO3-SARP-derived plasmids containing the aRNA library, obtained by subcloning. A single transformation colony is then plated on a yeast tryptone (YT) agar plate containing chloramphenicol (80 ug/ml) and incubated at 42° C. The pKO3-SARP plasmid confers chloramphenicol resistance but is temperature sensitive and thus, cannot replicate at 42° C. Only the cells in which the plasmid has integrated into the chromosome will be able to grow in presence of the antibiotic. A single colony is then plated onto a YT plate containing kanamycin (50 ug/ml) and sucrose (5% w/v), and incubated at 30° C., at which a plasmid can replicate. The host plasmid also contains the Bacillus subtilis sacB gene, whose gene product kills E. coli cells grown in the presence of sucrose. At 30° C., cells that contain a chromosomal copy of the plasmid cannot grow efficiently. Thus, the colonies that do grow result from a second recombination event in which the plasmid containing the exchanged host 5S rRNA gene has been excised from the chromosome. The resulting cell line is further transformed with plasmid pCP20. This plasmid is also temperature sensitive for replication as for FLP expression and at 42° C. both expresses Flp recombinase and ceases to replicate, resulting in the excision of the kanamycin gene from between the FRT flanking sites in the chromosome and loss of the pCP20 plasmid.

### Example 3

### PCR, Cloning, and Sequencing for Verification of Random Library Strains

To verify that our randomized aRNA library has been inserted into the E. coli chromosome several primers that have been described in Ammons et al. will be used. Primers A (CCCGAGACTCAGTGAAATTG) (SEQ ID NO:1), B (CCCAAGAATTCATATCGACGGC) (SEQ ID NO:1), C (CCCAAGCTTCGCTACTGCCGCCAGGCA) (SEQ ID NO:3), and D (TCCCCCGGGAGTAGGGAACTGCCAGGCAT) (SEQ ID NO:4) are nonspecific and hybridize to orthologous regions present in all seven rRNA operons. Primer E (GGCTCTCTTTCAGACTTGGG) (SEQ ID NO:5) is specific for rRNA operon H. PCR amplification using primers A and E will allow for the discrimination between an aRNA gene insertion and a wild type 5S rRNA gene and this will be evident by standard electrophoretic analysis. The amplified sequence would then be cloned using the TOPO TA system (Invitrogen) to be subsequently sequenced with the expectation that the randomized region will generate many indeterminate base-calls or “Ns” by sequencing.

### Example 4

### Quantification of Library Expression Levels

To quantify the relative amount of the aRNA library being expressed, total RNA from cells in log-phase growth using a standardized Trizol™ reagent (Invitrogen) or similar “home brew” version will be isolated. See Chomczynski P, Sacchi N: Single Step Method of RNA Isolation by Acid Guanidinium Thiocyanate-Phenol-Chloroform Extraction. Anal Biochem 1987, 162:156. Following a 30 nt deletion and 50 nt insertion of randomized sequence, the final “5S-like” RNA pool for in vivo selection will be ˜140 nt long. This is 20 nt longer than 5S rRNA, the nearest major RNA species to be isolated by the Trizol method. It will therefore be straightforward to separate this RNA library of interest from the native RNAs by standard electrophoretic methods and quantify the relative expression of the library. The relative expression will be characterized at various times in the growth cycle of the cells and for various growth conditions.

### Example 5

### In Vivo Selection of Nickel Aptamers

While nickel is referred to as a heavy metal, it is relatively safe to work with. Novel genomically modified strains expressing randomized 50-mer ribosomal-inserts, will be cultured in the presence of increasing NiCl₂ concentrations. Strains capable of growth within at least 10 mM Ni²⁺ are expected to evolve. This represents a heavily contaminated water stream at over 100 parts per million (ppm) levels or 6.022×10¹⁸ nickel atoms/ml. At a minimum a culture containing ˜1×10⁹ cells/ml each containing ˜50,000 ribosomes, is expected to impart this level of tolerance. If 1/7 of these ribosomes contain an aptamer the culture will contain ˜7×10¹² aptamers/ml. Thus, at 10 mM, it is likely that the nickel ions are in excess to the evolved aptamer ligands, yet it is apparent from these numerical estimates that ribosomal aptamers are being produced in large numbers and impart increased fitness to the organism in the presence of contaminant.

### Example 6

### In Vivo Selection of Malachite Green, Luciferin, and O-estradiol Aptamers

Malachite green is a widely used triphenylmethane dye with known ability to cross into the cell cytoplasm. Further, Babendure, et al. have selected aptamers in vitro against malachite green and related molecules which, when bound to the dye, cause a tremendous fluorescence enhancement (greater than 2300-fold) that is readily detectable by spectroscopy and/or fluorescence microscopy. Whether or not this fluorescence enhancement occurs when bound to our in vivo selected aptamers, malachite green should be easily detectable both inside and outside of cells. This feature will be especially useful in demonstrating that the novel strains of the present invention improve sequestration of contaminants inside of cells. Malachite green is also interesting as an organic molecule of similar molecular weight as that of many problematic water contaminants, and variants of the dye with halogen and other substitutions are available. Finally, the compound itself is a known carcinogen and is used as an antimicrobial agent in aquaculture in some parts of the world. Development of ribosomal aptamers to malachite green may therefore have intrinsic value in its own right.

Luciferin is the substrate of the enzyme luciferase. Luciferin is also similar in molecular weight to many toxins, pharmaceuticals, pesticides, and hormones. Ribosomal-aptamers to luciferin, developed will aid in assaying for its sequestration within E. coli by the use of luciferase, the well known light-generating firefly enzyme.

β-estradiol is the major estrogen secreted by the pre-menopausal ovary. Exposure to estradiol increases breast cancer incidence and proliferation. Increasing evidence is mounting that estrogens and mimics are accumulating in the environment with detrimental effects on a variety of plants and animals, including humans.

First, the toxic limits of malachite green, luciferin, and β-estradiol for unmodified strains of E. coli will be determined. The random library strain developed supra will be cultured under increasing concentrations of the three model contaminants. Any culturing scheme in which the successive generations of cells are exposed to increasing contaminant pressure (concentration) should be suitable for the purposes of directing evolution of the random library to specific aptamer sequences. Especially in the case of malachite green, some significant portion of total contaminant is expected to be sequestered in the inner membrane (peptidoglycan) region of E. coli. The dye has been routinely used to stain bacterial endospores within cells of species such as Bacillus anthracis. Hence, some fraction of the malachite green will be available in the cytosol and some bulk concentration will induce selective pressure to develop aptamers to the compound. As with malachite green, it is expected that at some high concentration, growth will be inhibited thereby ensuring that the molecule is available in the cytosol and selective pressure is applied to the random ribosomal library.

### Example 7

### To Demonstrate Improved Sequestration of Contaminants from Water Using Our Novel Bacterial Strains Containing Ribosomal Aptamers

Having evolved new ribosomal-aptamers to model water contaminants in vivo and characterized their binding affinity in vitro, the ultimate utility of our approach for facilitating contaminant clearance from water streams will be demonstrated. The assumption is that in most applications, sequestration of trace molecular contaminants (whether they are subsequently degraded or not) within cells will facilitate their removal either by mechanical filtration of cells or settling. In contrast to the in vitro experiments involving only purified aRNA, these experiments will use whole E. coli cells containing ribosomal-aptamers. To demonstrate the enabling nature of ribosomal-aptamers, several qualitative and quantitative partitioning experiments using model contaminant targets will be performed.

### Mechanical Filtration and Nickel Quantitation for Nickel Partitioning Measurement

To demonstrate sequestration of the heavy metal ion Ni²⁺, the nickel aptamer strain developed above will be cultured in the presence of increasing concentrations of NiCl₂. Immediately following culture to log-phase growth (OD₆₀₀≈1.0) the cell suspensions will be mechanically filtered using commercially available 0.2 μm syringe filters. The nickel will be then quantified in the filtrate as described by spectrophotometry. If necessary for reliable spectrophotometric quantitation, nickel calibration curve in the optically-clear culture medium M9 and culture strains in that medium (with any necessary supplementation) will be developed. As a control, the same experiments using standard strains of E. coli with no ribosomal-aptamers will be performed. Any nickel retention by the control strain will be “subtracted off” of the results for the nickel aptamer strain prior to calculation of partitioning coefficients. Partitioning values will be calculated in triplicate for approximately 7 nickel concentrations.

### Centrifugation and Supernatant Analysis for Malachite Green, Luciferin, and ¹⁴C-Estrogen Partitioning Measurement

Similar to the above nickel experiment, E. coli containing aptamers and mechanical filtration to mechanically sequester model organic contaminants will be used. One concern, however, is that some amount of these organics might bind to the syringe membrane filters (typically PVDF, however we may investigate alternatives). To avoid this complication, the cell suspension will be partitioned by centrifugation. The supernatants will then be analyzed for malachite green, luciferin, and β-estradiol as described supra. As in the nickel partitioning determination, baseline sequestration of these organics using a control strain (which is likely to be more significant due to hold-up in the inner membrane of E. coli) will be subtracted before determination of partitioning coefficients.

### Fluorescence Microscopy of Increased Malachite Green Sequestration

Malachite green aptamer strains and control strains of E. coli will be bathed in several concentrations of malachite green, cells will be affixed to slides using standard methods, and examined using either fluorescence microscopy or fluorescence imaging. To decrease, background fluorescence (if necessary) cells will be cultured on membranes (Neogen, Inc) placed on agar containing malachite green. The membrane will be transferred to agar containing activated carbon. Excess malachite green will therefore be “destained” with the expectation that the aptamer-containing cells will fluoresce with much more intensity than the control strain.

### Example 8

### To Demonstrate Collection of aRNAs from Host Cell Culture

Briefly, E. coli cell paste was resuspended (approximately 10 ml/liter culture) in final concentrations of 100 mM Tris-Acetate, 10 mM Na₂-EDTA, 1% (w/v) SDS, 5 molar formamide. The E. coli were transformed to include an aRNA with an shRNA insert incorporated into a 5S rRNA deletion mutant. The suspension was incubated with gentle shaking at 37° C. for 20 min such that the cell membranes are lysed and RNA is released. An equal volume of 3 M potassium acetate (pH 4.8) was added followed by gentle shaking for additional 10 min, and a precipitated material (DNA, protein, cell debris) was spun down. The cleared supernatant was transferred to a new tube, and RNA was then precipitated by conventional ethanol precipitation. The RNA pellet was then selectively resuspended in 3M sodium acetate (pH 5.0). At this stage, a significant part of the pellet remained insoluble due to the presence of denatured proteins, RNA was solubilized completely. The cleared supernatant (low molecular weight fraction not containing 16S and 23S rRNA, was then ready for subsequent processing. Similar scalable protocols for RNA fractionation using polyamine compaction, affinity chromatography and/or any other appropriate method may also be utilized to isolate RNAs based on size.

### Example 9

### To Demonstrate Excision of an Insert Utilizing DNAzymes

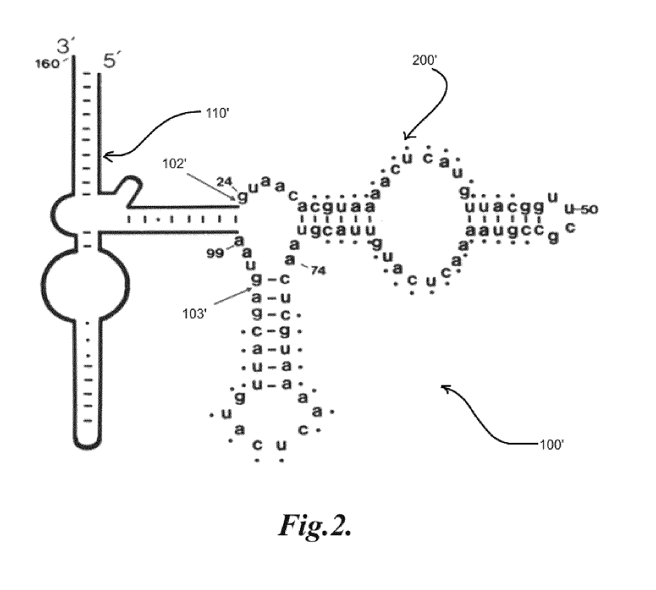

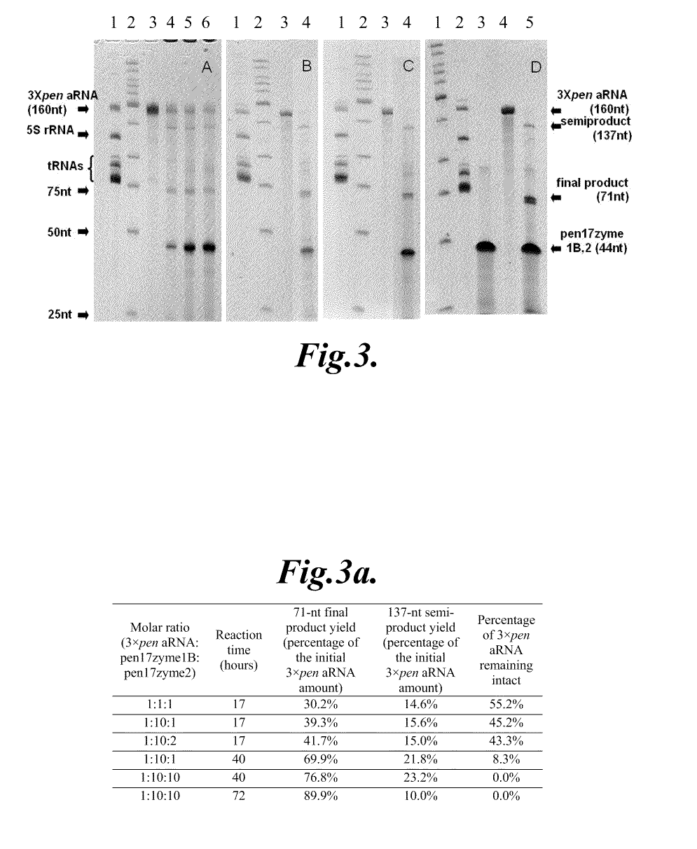

A pair of DNAzymes, “Pen17zyme1B” and “Pen17zyme2”, were designed to cleave an aRNA substrate, as shown with aRNA 100′ in FIG. 2, in two places 101′, 102′, thereby liberating the desired insert sequence 200′ from the carrier portion 110′. Some examples of cleavage conditions for the DNAzymes are shown in FIG. 3, illustrating a denaturing 8% PAGE gel showing aRNA cleavage by pen17zyme1B and 2. Incubation of aRNA (160 nt) with the two DNAzymes yielded a 71 nt final product and 137 nt intermediate. Panel A shows: (1) E. coli JM109(DE3)/pCP3×3 total RNA, (2) ladder, (3) 3×pen aRNA, (4-6) after DNAzyme cleavage. Panels B/C: (1-4) same as Panel A. Panel D: (1) ladder, (2) pCP3×3 total RNA, (3) pen17zyme1B,2, (4) 3×pen aRNA, (5) after DNAzyme cleavage. In FIG. 3, various substrate to enzyme ratios and reaction times were utilized: Panel A: lane 4, aRNA: pen17zyme1B: pen17zyme2=1:1:1, 23° C. for 17 hours; lane 5, aRNA: pen17zyme1B: pen17zyme2=1:10:1, 23° C. for 17 hours; lane 6, aRNA: pen17zyme1B: pen17zyme2=1:10:2, 23° C. for 17 hours. Panel B: lane 4, 3×pen aRNA: pen17zyme1B: pen17zyme2=1:10:1, 23° C. for 40 hours. Panel C: lane 4, 3×pen aRNA: pen17zyme1B: pen17zyme2=1:10:10, 23° C. for 40 hours. Panel D: lane 5, 3×pen aRNA: pen17zyme1B: pen17zyme2=1:10:10, 23° C. for 72 hours.

For example, in FIG. 3, Panel D, Lane 5 shows a ratio of aRNA substrate:pen17zyme1B:pen17zyme2=1:10:10, at 23° C. for 72 hours to yield a desirable amount of final product. Panel D, Lane 4 of FIG. 3 shows that an artificial, ribosomal-like RNA containing an arbitrary insert sequence can accumulate to high levels and be pre-purified on a quantitative scale. Lane 5 of Panel D, FIG. 3 then demonstrates that the insert sequence (arrow to final product (71 nt)) was specifically excised by a pair of DNAzymes in high yield. In general, aRNA substrate and DNAzymes were incubated together in 50 mM MOPS pH 7.2, 500 mM SpermineHCl at 90° C. for 2 min followed by 23° C. for 10 min. Appropriate volumes of stocks were added to achieve final concentrations of 125 mM KCl, 500 mM NaCl, 7.5 mM MgCl₂, 15 mM MnCl2. The addition of the divalent cation salts was used to initiate the reactions at 23° C.

FIG. 3a shows a table of the yield of various excision reactions using DNAzymes under varying conditions.

### Example 10

### To Demonstrate Excision of an Insert Utilizing RNase III

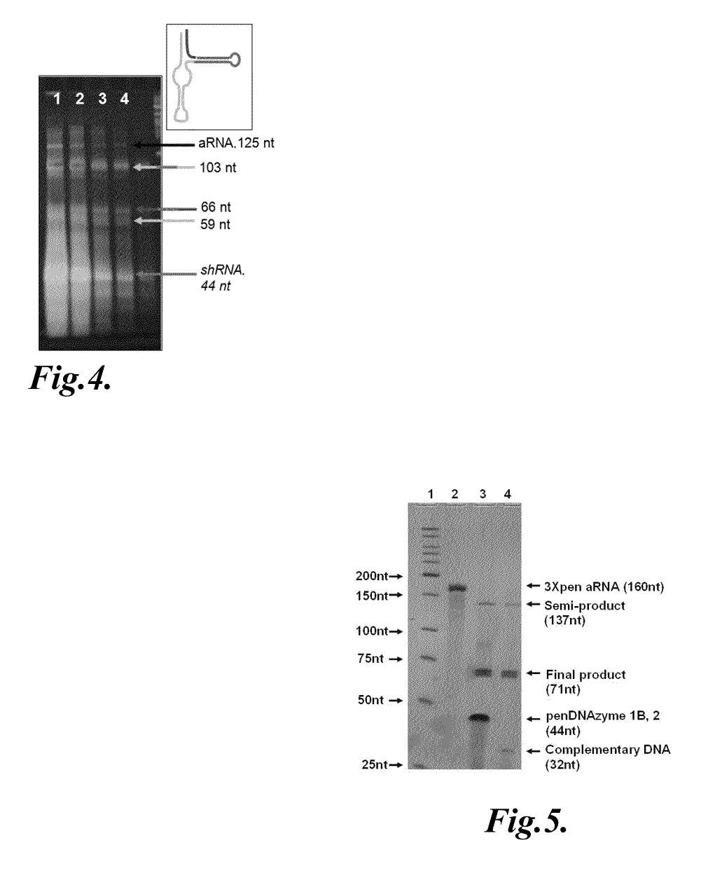

RNase III recognizes stretches of double-stranded self-complementarity in RNA and then cleaves dsRNA or hairpin RNA after approximately every two helical turns, which may generally result in homogeneous length of the resulting products. RNase III was utilized to process in vitro transcribed versions of an aRNA substrates as shown in FIG. 4. Lanes 1-4 are a 2× dilution series of the same reaction. As shown in, for example, Lane 1, a very large amount of shRNA hairpin product was released by RNase III treatment.

### Example 10

### To Demonstrate Excision of an Insert Utilizing RNase H

RNase H may generally be utilized to specifically digest an unwanted RNA scaffold. RNase H generally specifically digests RNA in DNA:RNA hybrids and may thus be utilized generally (regardless of insert sequence) to digest the surrounding carrier RNA of an aRNA. The DNA for hybridizing to the carrier RNA portion of the aRNA was biotinylated and was immobilized or removed post-reaction by high affinity streptavidin beads. FIG. 5 shows an example of a gel showing the products of RNase H digestion of an aRNA. Lanes in the denaturing 8% PAGE are as follows: Lane 1, RNA marker; Lane 2, 3×pen aRNA; Lane 3, RNA mixture after the cleavage reaction; Lane 4: RNA eluted from streptavidin beads carrying biotinylated 32-mer DNA oligo bioantiPEN, complementary to the 71 nt final product.

It will be appreciated by those of ordinary skill in the art that the present invention can be embodied in other specific forms without departing from the spirit or essential character hereof. The present description is therefore considered in all respects to be illustrative and not restrictive. The scope of the present invention is indicated by the appended claims, and all changes that come within the meaning and range of equivalents thereof are intended to be embraced therein.

1. An expression vector comprising:
a chimeric gene encoding a gene product comprising a selective nucleic acid within a carrier nucleic acid, wherein said selective nucleic acid is capable of binding to or affecting at least one target molecule;
wherein said expression vector when transfected into a host transcribes said chimeric gene.

2. The expression vector of claim 1, wherein said vector further comprises at least one of a functional promoter, a selection marker or a marker for selective induction.

3. The expression vector of claim 2, wherein said at least one selection marker comprises an antibiotic resistance marker.

4. The expression vector of claim 2, wherein said promoter is a T7 RNA polymerase or a ribosomal RNA promoter.

5. The expression vector of claim 1, wherein said selective nucleic acid comprises at least one of a short interfering RNA (siRNA); a micro RNA (miRNA); a short hairpin RNA (shRNA); an aptamer; a ribozyme; an aptazyme; a riboswitch; an aptamer inhibitor; an antisense nucleic acid; a probe library, a diagnostic probe; a precursor thereof or a combination thereof.

6. The expression vector of claim 1, wherein said carrier nucleic acid is selected from the group consisting of rRNA, tRNA, RNAase P, small nuclear RNA (snRNA), small nucleolar RNA (snoRNA), efference RNA (eRNA) and tmRNA.

7. The expression vector of claim 1, wherein said at least one target molecule is a nucleic acid.

8. The expression vector of claim 7, wherein said nucleic acid is a messenger RNA (mRNA).

9. The expression vector of claim 8, wherein said selective nucleic acid participates in an RNA interference (RNAi) mechanism.

10. The expression vector of claim 1, further comprising at least one self-excising RNAzyme sequence.

11. The expression vector of claim 1, further comprising at least a sequence complementary to the hybridization sequence of a DNAzyme.

12. An isolated cell comprising:
at least one selective nucleic acid sequence incorporated into a genomic DNA encoding a non-coding nucleic acid, wherein said selective nucleic acid binds to or affects a target molecule.

13. The cell of claim 12, wherein said selective nucleic acid is incorporated into said non-coding nucleic acid by homologous recombination.

14. The cell of claim 12, wherein said non-coding nucleic acid is selected from the group consisting of rRNA, tRNA, RNAase P, small nuclear RNA (snRNA), small nucleolar RNA (snoRNA), efference RNA (eRNA) and tmRNA.

15. The cell of claim 12, wherein said cell is a prokaryotic cell or a eukaryotic cell.

16. The cell of claim 12, wherein said selective nucleic acid comprises at least one of a short interfering RNA (siRNA); a micro RNA (miRNA); a short hairpin RNA (snRNA); an aptamer; a ribozyme; an aptazyme; a riboswitch; an aptamer inhibitor; an antisense nucleic acid; a probe library, a diagnostic probe; a precursor thereof or a combination thereof.

17. The cell of claim 12, wherein said at least one target molecule is a nucleic acid.

18. The cell of claim 17, wherein said nucleic acid is an mRNA.

19. The cell of claim 12, wherein said selective nucleic acid participates in an RNAi mechanism.

20. A method for generating amounts of selective nucleic acids comprising:
generating a library of nucleic acid sequences encoding selective nucleic acid capable of binding to or affecting at least one target molecule;
incorporating said nucleic acid sequences in at least one carrier nucleic acid in a cell;
culturing said cell to achieve a cell population; and
purifying selective nucleic acids incorporated into carrier nucleic acids from said cell population.

21. The method of claim 20, wherein said cell is selected from the group consisting of E. coli, Staphylococcus, Bacillus, Pseudomonas, Citrobacteia, Klebsilla, and Rhodococcus or Saccharomyces (yeast).

22. The method of claim 20, wherein purifying said selective nucleic acids incorporated into carrier nucleic acids comprises at least partially lysing cells of said cell population and at least partially selectively purifying nucleic acids of a certain size range.

23. The method of claim 22, further comprising excising said selective nucleic acids from said carrier nucleic acids.

24. The method of claim 23, wherein said excising comprises using RNases, DNAzymes, chemical scissors or a combination thereof.

25. The method of claim 21, wherein said cell is selected or modified for deficient or inducible activity of at least one RNase.
